(19) United States
(12) Patent Application Publication
(10) Pub. No.: US 2026/0314974 A1
(43) Pub. Date: Oct. 15, 2026

(54) Device for Detecting Magnetic Signals Generated by a Beating Heart

(71) Applicant: Robert Bosch GmbH, Stuttgart (DE)

(72) Inventors: Florian Dolde, Stuttgart (DE); Frederik Schaal, Reutlingen (DE); Andreas Brenneis, Renningen (DE); Timo Weggler, Ulm (DE); Florian Krist, Stuttgart (DE); Riccardo Cipolletti, Geislingen An Der Steige (DE); Janine Riedrich-Moeller, Leonberg-Warmbronn (DE); Eckhard Wehrse, Stuttgart (DE)

(21) Appl. No.: 19/108,499

(22) PCT Filed: Aug. 18, 2023
(86) PCT No.: PCT/EP2023/072824

§ 371 Date: Mar. 4, 2025

(30) Foreign Application Priority Data
Sep. 9, 2022 (DE) 10 2022 209 430.1

(51) Int. Cl. A61B 5/243; A61B 5/00

(52) U.S. Cl. CPC A61B 5/243; A61B 5/7203; A61B 5/7271; A61B 2562/046

(57) ABSTRACT

A device is for detecting magnetic signals generated by a beating heart. The device includes at least two nitrogen-vacancy center, NV, magnetometer units, which are arranged in a geometric arrangement relative to one another and are connected to a signal processing unit. The device is configured to detect a magnetic field strength and field direction using each of the at least two NV magnetometer units. The device uses the signal processing unit to determine at least one field vector having an effective magnetic field strength and an effective field direction from the magnetic field strengths and field directions detected by the at least two NV magnetometers.

The present invention relates to a device for detecting magnetic signals generated by a beating heart

### BACKGROUND OF THE INVENTION

Optically pumped or diamond-based quantum sensors based on NV centers are particularly suitable as sensors for measuring very small magnetic field strengths. DE 10 2022 204 526.2 describes a magnetometer that utilizes optically pumped and optically detected magnetic resonance (ODMR). It is thereby exploited that, under the influence of an external magnetic field, the energy levels of certain spin states of unpaired electrons are split, the so-called Zeeman effect. The splitting of the energy levels results in changed transitions during relaxation from excited states, which can then be measured, for example, by optical excitation and frequency-dependent detection of the resulting fluorescence radiation or by observation of optical properties such as the absorption of light. From the measured optical parameters, the magnetic field strength may then in turn be inferred.

### DISCLOSURE OF THE INVENTION

According to the invention, a device for detecting magnetic signals generated by a beating heart is proposed with the features of claim 1. Advantageous configurations are the subject matter of the dependent claims and the following description.

A magnetocardiogram (abbreviated MCG) is the recording and representation of the magnetic field of the heart that arises from the electrophysiological activity of the myocardial cells. In the context of the invention, a contactless, passive approach for long-term monitoring of the human heart with high resolution is presented. This is determined by nitrogen vacancy magnetometers (so-called NV magnetometers) in a geometric arrangement for vector gradiometry.

In particular, a device for detecting magnetic signals generated by a beating heart is now presented, which comprises at least two NV magnetometer units arranged in a geometric arrangement with respect to each other and connected to a signal processing unit, wherein the device is designed to detect a magnetic field strength and field direction by means of each of the at least two NV magnetometer units to detect a magnetic field strength and field direction, and by means of the signal processing unit to determine at least one field vector comprising an effective magnetic field strength and an effective field direction from the magnetic field strengths and field directions detected by means of the at least two NV magnetometer units. Both a wireless and wired connection are provided between the sensors and the signal processing unit. Such a device may also be referred to as a magnetocardiograph.

Diamond NV magnetometers are based on reading out magnetic resonances from specific defect centers in diamond, in particular nitrogen vacancies (NV), which occur as impurities in the carbon lattice of diamond and can also be introduced in a targeted manner. If the NV center is optically excited in the ground state, e.g., by radiating in a pump laser beam having a suitable wavelength (in this case in the green wavelength range, e.g., at 532 nm for off-resonance excitation), the electrons are lifted from the triplet ground state to the excited triplet state and relax while emitting fluorescence light in the red wavelength range at 650-800 nm (637 nm=zero phonon line). Since the probability of non-spin-conserving transitions from the spin state is higher when the spin quantum number mₛ=±1, continuous excitation pumping ensures that the NV centers are largely hyperpolarized in the spin state mₛ=0.

There is an energy difference between the mₛ=0 and mₛ=±1 spin states in the ground state, which in this case is about 2.87 GHz. Thus, if microwave radiation is radiated into the diamond in addition to the optical excitation, there is a dip in the red fluorescence at this resonance frequency of 2.87 GHz, because the spin-polarized electrons are lifted by the microwave field from mₛ=0 to the mₛ=±1 ground state and excited from there by the pump light to the mₛ=±1 excited state. From there, however, primarily non-radiative transitions and weak infrared fluorescence transitions occur via the singlet state, and the fluorescence in the red range ceases.

If an external magnetic field is present, the so-called Zeeman effect leads to the splitting of the otherwise energetically equal mₛ=±1 triplet levels into energetically equidistant Zeeman levels. Plotting the fluorescence against a frequency spectrum of the microwave excitation then reveals two dips in the fluorescence spectrum, the frequency spacing of which is linearly proportional to the magnetic field strength of the external magnetic field. The magnetic field sensitivity is primarily defined by the minimally resolvable frequency shift and can reach 1 pT/√Hz or less. Because the nitrogen vacancy center in the single-crystal diamonds has four possible ways to position itself in the crystal lattice, the presence of a directed magnetic field causes the nitrogen vacancy centers present in the crystal to react differently to the external magnetic field depending on their position in the crystal. As a result, four pairs of fluorescence minima may appear in the spectrum, from the shape and position of which both the magnetic field strength as an amount and the direction of the external magnetic field are clearly determinable. In order to enable vectorial magnetic field measurements, the device comprises a means for generating a substantially homogeneous bias magnetic field in the region of the magnetometer units or their sensor media, wherein the bias magnetic fields of different NV magnetometer units are different. It can be a Helmholtz coil arrangement, wherein at least the sensor medium is arranged inside the Helmholtz coil arrangement (each magnetometer unit has its own bias field). It can also be other equipment such as a simple coil, a long coil, permanent magnet solutions such as in a Hallbach array, etc.

By using a number of vector magnetometer units in a geometric arrangement and gradiometer circuitry, these magnetometer units have a different orientation to the magnetic field of the heart. The gradiometer circuitry, i.e. essentially vector arithmetic of the measured data, can be used to determine the position and strength of the magnetic field exciter (heart). Since the much stronger background field is essentially the same in both magnetometer units (same strength and orientation), it can be eliminated. This eliminates the need for magnetic shielding so that magnetic field measurement can be carried out in everyday environments. Another advantage over conventional gradiometer arrangements with one-dimensional magnetometers is the compact design of the gradiometer arrangement, since no remote reference magnetometer is needed. Accordingly, the invention is particularly suitable for non-shielded measurement of weak magnetic fields. Technical details for gradiometer solutions that can also be used in the context of the present invention are disclosed in DE 102022201690.4 and are to be incorporated herein.

Cardiac signals have a magnetic signature at a distance of some cm with an amplitude of (only) single digit to double digit picotesla (pT), whereas, for example, the Earth's magnetic field in central Europe is approximately 50 μT (microtesla), i.e., it is more powerful by a factor of 10⁶. However, even such small field strengths are resolvable with a high level of precision for a long period of time with the proposed technology.

High resolution detection of the accurate heart signal can be used to detect a variety of diseases such as permanent atrial fibrillation and paroxysmal atrial fibrillation. Thus, a heart attack and subsequently a stroke (particularly after an unrecognized heart attack) can be prevented. Further, the invention is suitable for early detection of an ST segment elevation infarction, a different kind of elevation infarction, a pulmonary embolism, an AV nodal reentry tachycardia, ventricular extrasystoles, as well as very rare pathogenic diseases such as arrhythmogenic right ventricular tachycardia, which can otherwise only be detected by gene sequencing.

Another particular advantage of the NV sensor technology is the direction or vector information. In contrast to other technologies, this is intrinsic in NV sensor technology. Thus, interferences do not need to be introduced through modulation techniques and/or less favorable projections do not need to be used, nor do multiple separate sensors need to be used. The vector and gradiometry information is thus available at the exact same location (diamond size, i.e. single digit mm\hat{ }3 and below) and not separated by a few cm to many cm as with other technologies. With NV magnetometer units, which can determine not only the field strength but also the direction of the magnetic field, improved suppression of a background field and thus better detection of signals with superimposition of strong interference signals are enabled. This is based on two mechanisms of action: firstly, the signal is not so susceptible to tilting of the sensors with respect to one another (in a standard gradiometer application, tilting of the 1D magnetomer leads to a different projection of the measurement or interference field), and this can be compensated for, which is particularly relevant for non-rigid designs combined with localization, e.g. in a mattress. Second, the interference field may have a different spatial direction than the field being measured. This facilitates the separation of the signal and background.

The directional information can be used to record channels equivalent to ECG channels, especially when gradiometer arrangements are used that consist of at least two NV magnetometer units with different main planes. A vectorial and/or scalar difference of various NV magnetometer units in an arrangement can be formed to detect and eliminate background noise. The formation of time derivatives of the signals can also be used. The vector information allows precise (not only projection, but direct) correction/separation of background/interference signals. This solves the problem that, in the case of classic gradiometers consisting of one-dimensional magnetic field sensors, these must be precisely aligned with each other at an angle. With the vector information, this is no longer necessary because the interference fields are completely known.

It is also possible to utilize the gradiometric directional differences or the various vector signals, or both, to generate information equivalent to different ECG channels (directionally and spatially resolved signals from the heart). In one embodiment, the signal processing unit is designed to determine a plurality of field vectors, each comprising a magnetic field strength and a field direction, from the magnetic field strengths and field directions detected by means of the at least two NV magnetometer units.

In particular, spatial resolution of the heart can be accomplished by multiple individual magnetometer units of an arrangement in proximity to the heart, wherein a straight projection of the heart onto the arrangement covers multiple NV magnetometer units. The spatial 3D course of the magnetic field (several field vectors) of the heart is detected. Depending on the implementation, actual 2D and 3D images of the heart are obtained, enabling much more extensive analyses to be carried out than before.

After the data has been processed, the signal is analyzed. In particular, attention is paid to certain terms that are indicators of certain problem signals or of a healthy signal. If these indicators deviate from the norm to a certain extent, a fault signal can be emitted, for example, which can trigger an alarm or forward the signal to a doctor.

Only an accurate resolution of the heart signal allows for this disease detection. Namely, in the case of very noisy or poorly resolved signals, the displacements of the various PQRST complexes of the heart compared to each other or over time, fluctuations in their amplitude, deformations or minor disorders are not detectable. However, this resolution of these criteria are important factors because the abovementioned problems can lead to mixing up complexes (e.g. interpreting an elevated and delayed T-wave as an R-wave, which is however common in a “healthy” heart), and to false alarms. In particular, the vector information also allows the heart's position to be localized, thus enabling the source to be determined and data from the correct direction to be explicitly evaluated. This can also be used in particular to measure individual parts of the heart or other objects and to specifically assign signals to them. Thus, the information content is higher compared to ECG, since the heart is spatially resolved and thus various abnormalities can be specifically assigned to physical areas. This allows more accurate diagnoses. In addition, there are signals that are only visible in the MKG but not in the ECG, especially in these directional signals, which provide additional information, e.g. when one ventricle of the heart “lags behind” the other. The MKG signal is particularly revealing here, since the artifacts appear in different directions and at different locations, allowing them to be precisely identified.

Another advantage of the NV sensor system is its size, especially the sensor medium. For the application, the active measurement volume should be small compared to the object (heart) to be measured, otherwise integration will occur over large portions of the signal due to the surface coverage, and thus the signal may disappear because the integral is zero. The smaller the active measurement volume is compared to the heart, the better the signal detection will be. NV sensor technology has a very small active sensor volume. This capability for creating small designs also allows the sensors to be used in a geometric arrangement. In particular, very high resolution arrangements are possible due to the very small active sensor volume.

A sampling rate which is higher than the cardiac signal, in particular greater than 50 Hz, is required for the application in order to resolve the signal. A range of 200 Hz to 400 Hz is considered to be particularly advantageous. Higher is always better for resolution, however this increases the requirements for sensitivity.

Further advantages and embodiments of the invention will emerge from the description and the accompanying drawings.

The invention is shown schematically in the drawings on the basis of exemplary embodiments and is described hereinafter with reference to the drawings.

### BRIEF DESCRIPTION OF THE DRAWINGS

FIG. 1 shows in a schematic block view the essential components of an NV-center magnetometer as may be used in the context of the invention.

FIG. 2 shows in various figures a) through c), each in a schematic block view, possible geometric arrangements of NV magnetometer units of a device for detecting magnetic signals according to one embodiment.

FIG. 3 shows a schematic block diagram of a measurement principle for NV vector gradiometry, as it may be used in an embodiment of the invention.

### EMBODIMENT(S) OF THE INVENTION

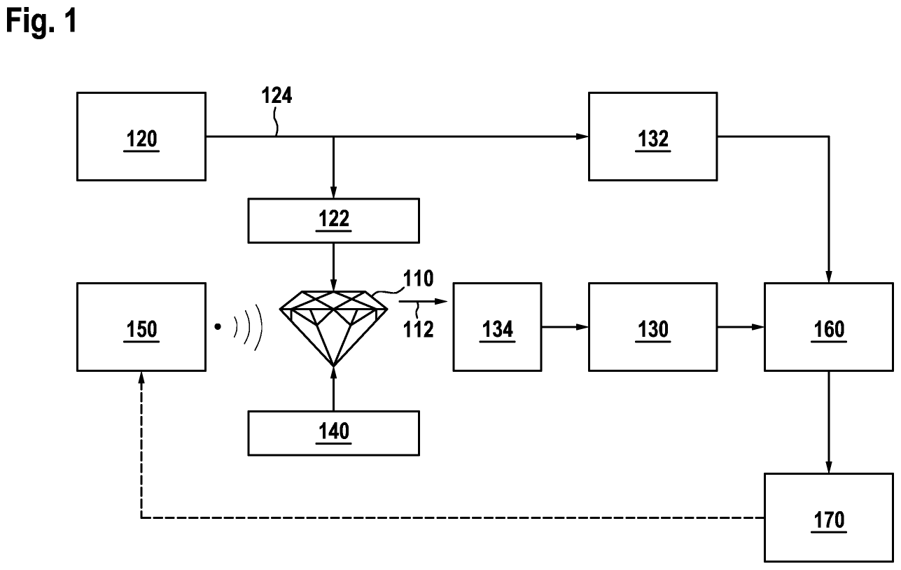

FIG. 1 shows a diagram of the essential components of an NV-center magnetometer. First, a diamond 110 with nitrogen vacancies (NV) is present as the sensor medium. The optical excitation of the NV centers is achieved by a suitable light source 120, such as an LED or a pump laser. Here, for example, a frequency-doubled Nd:YAG laser or semiconductor laser in the green range of about 510-532 nm, e.g. at 532 nm, is suitable for off-resonance excitation. Alternatively, LEDs in suitable wavelength ranges may also be used. Depending on the arrangement, the light of the light source 120 may be radiated via suitable optical elements 122 such as mirrors, beam splitters, focusing optics such as lenses, and optionally via fiber optic elements in the diamonds 110. In addition, the excitation light may be irradiated continuously or in a pulsed manner by the laser such that, for example, time windows for interference-free fluorescence light measurement are kept clear.

Furthermore, a microwave source 150 may be present in the magnetometer capable of generating an electromagnetic field across a bandwidth that sufficiently covers the desired resonance frequency in the sensor medium, e.g., in the range of the NV centers in the diamonds 110. A microwave resonator structure may be used to homogeneously distribute the generated microwaves across the volume of the sensing range in the diamond. The resonator structure or microwave source 150 is preferably tuned for the frequency of the electron spin resonances. To enable vector magnetometry, an additional static bias magnetic field 140 is generated. As a result, the measurement becomes intrinsically vectorial. Different spatial directions are used in the crystal structure for this purpose. For example, to generate such a magnetic field 140, a Helmholtz coil is suitable, in which a substantially homogeneous magnetic field can be generated in a limited range by means of a pair of coils.

The resulting fluorescent light 112 from the diamond 110 may in turn be directed via suitable optical elements 134 such as optical filters, beam splitters, lenses, and/or fiber optic elements to a first photodetector 130 that is sensitive at least in the range of the fluorescence wavelength. The first photodetector 130 may also be arranged directly on the diamond 110. A second photodetector 132 is arranged to detect at least a portion of the excitation light of the light source 120, which may be decoupled by, for example, a beam splitter, a filter, or a partially permeable element. This excitation light detector signal 132 may be used as a reference signal to eliminate background signals, for example, by modulating the excitation light by way of a lock-in amplifier, and highlighting the resonance signal of interest. Additionally, or alternatively, this reference signal may be used to account for fluctuations in excitation light. Corresponding circuitry 160, such as a pre-amplifier, log amplifier, lock-in amplifier, signal filters, or others, is thus provided to receive the signals from the first and second photodetector and to pre-process the signals in an appropriate manner for further evaluation. Finally, the pre-processed fluorescence signal may be evaluated by a signal processing unit 170, e.g., with a suitable microcontroller or processor, to obtain the desired parameters of the detected magnetic field from the signal, in particular the magnetic field strength and the direction of the magnetic field.

Of course, such a device may also comprise further units not shown, such as communication units or interfaces to output measurement results. Such a device may also be advantageously integrated into an ASIC or FPGA.

In order to be usable in an everyday environment, magnetic fields not originating from desired weak sources are to be eliminated as far as possible from the measurement, in particular the Earth's magnetic field in the range of 10⁻⁵ Tesla (several microtesla). In contrast, cardiac magnetic fields range from 10 to 100 times 10⁻² Tesla (picotesla).

The elimination of the background magnetic fields may be achieved by a gradiometer arrangement for the magnetic field measurement, according to exemplary embodiments. Gradiometers are generally referred to as sensor units that are capable of detecting not only the field strength, but also the gradient of the field.

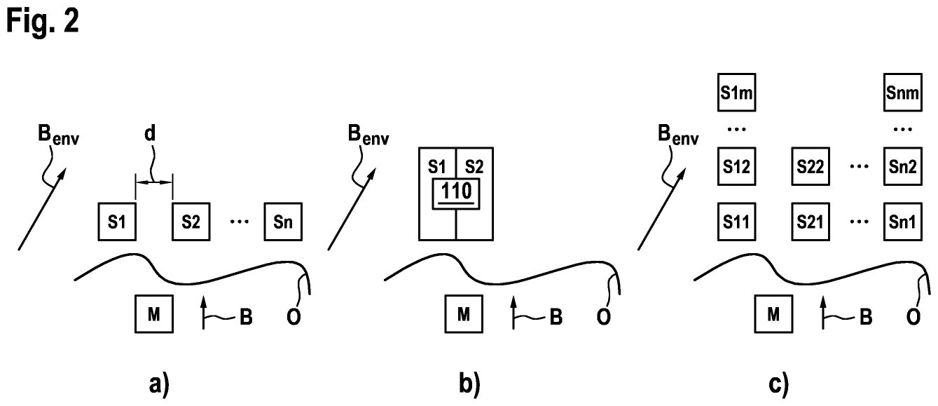

For this purpose, at least two individual magnetometer units S1, S2, . . . , Sn, S11, S21, . . . , Sn1, S12, S22, . . . , Sn2, S1m, . . . , SnmS11, S21, . . . , Sn1, S12, S22, . . . , Sn2, S1m, . . . , Snm can be used, which are arranged at spatially different locations. As an example, a sensor unit that uses two or more NV center magnetometers in a gradiometer arrangement is described below in conjunction with FIG. 2.

FIG. 2 shows possible geometric arrangements of NV magnetometer units of a device for detecting magnetic signals according to one embodiment in various figures a) through c). Figure a) shows a side view of an arrangement of at least two NV magnetometer units S1, S2, . . . , Sn in any arrangement with respect to one another in a plane (perpendicular to the drawing plane, i.e. only the first row is visible). Figure b) shows a side view of two NV magnetometer units S1, S2 whose sensor media are a section of the same diamond crystal 110. Figure c) shows a side view of a number (n by m) of NV magnetometer units S11, S21, . . . , Sn1, S12, S22, . . . , Sn2, S1m, . . . Snm in any three-dimensional arrangement. Further (not shown) layers join behind the drawing plane so that, overall, a type of cubic grid is formed. At least one NV magnetometer unit (not shown) lying in one of the rear layers, for example, is not arranged in the same plane (drawing plane) in which other NV magnetometer units S11, S21, . . . , Sn1, S12, S22, . . . , Sn2, S1m, . . . Snm are arranged.

Furthermore, M designates a signal source, here a heart, and O designates an optional surface (especially the skin of the body), which limits the accessibility to and of the magnetic field source M.

In embodiments of the inventions, more than two (but at least two) NV magnetometer units in total may form one gradiometer. With each additional NV magnetometer unit, the background field can be determined better and the location and strength of the exciter can be better separated from the background.

In other embodiments of the invention, two NV magnetometer units may also always form a gradiometer, wherein multiple gradiometers are then formed overall—depending on the number of NV magnetometer units—and the signal of interest is detected. An effective measurement signal can then be formed therefrom, in particular from the signal processing unit, for example by averaging, summation, etc.

A distance d between two NV magnetometer units S1, S2 . . . or more precisely their sensor media, corresponds to the distance between the locations where magnetic field measurements are taken simultaneously. As long as the distance between the measurement locations is relatively small, it can be assumed that the strength of an additional background magnetic field Bₑₙᵥ is approximately equal at both locations. In contrast, the weak magnetic field B of interest will decrease significantly with increasing distance from the magnetic field source M. Furthermore, the different orientation of the magnetic field of the exciter (e.g., the heart) allows for a clear separation from the background field even at close distances.

Thus, by arranging two NV magnetometer units at different distances and angles from the source or from the heart, the background field can be eliminated or determined by vector arithmetic, thereby determining the small magnetic field of interest and characterizing its source (location and orientation). This may be further improved by a remote magnetometer that is far enough away that the weak magnetic field of interest has dropped below the detection threshold. With such a configuration, local changes in the background field can be compensated for by the at least two near magnetometers. For this purpose, for example, two NV magnetometer units may be arranged one above the other in an axial gradiometer configuration, such that one NV magnetometer unit of a first layer forms a gradiometer with an NV magnetometer unit located below it in a second layer below. The background field can also be determined by means of a further NV magnetometer unit at a large distance, e.g. at least 1 m, from the two NV magnetometer units.

One concept of the invention is to measure the field with several NV magnetometer units vectorially and simultaneously from different positions. As long as this arrangement is in the vicinity of the signal source M to be measured, the field of the signal source is measured in different directions with the individual vector sensors. This distribution of the measured field vectors is characteristic and allows conclusions to be drawn about the source in terms of strength, position and orientation. In order to detect weak signals compared to the background, the assumption of a homogeneous background field over the geometric arrangement of NV magnetometer units (this is generally to be assumed here, since the distance between the source of the disturbance and the sensor medium is assumed to be much greater than that of the signal to be measured to the sensor medium). The individual field vectors can be calculated because the vector of the interference field can be assumed to be the same, and thus a simple vector arithmetic is sufficient to extract the signal vector. If a geometric arrangement is used, inhomogeneities in the background can also be compensated.

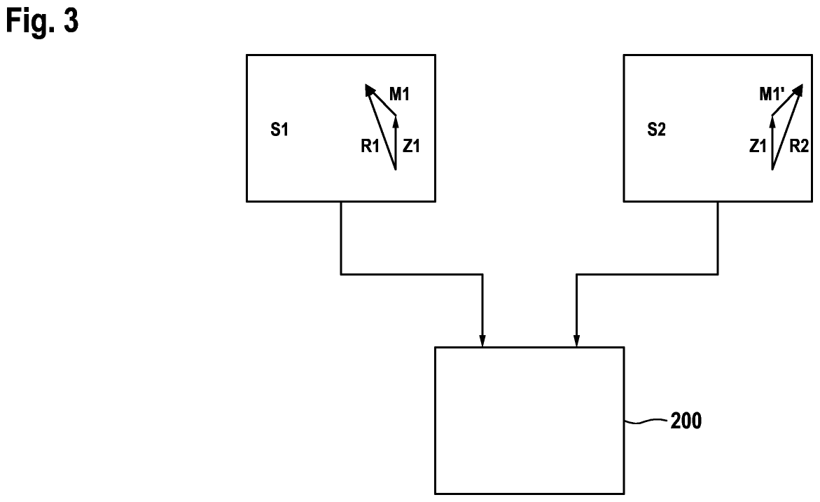

As shown in FIG. 3, the different measurement positions of the NV magnetometer units S1 and S2 determine the field vector in different directions. Since the background field Z1 is the same for both NV magnetometer units, this allows a calculation of the strength and orientation of the source. By measuring the signal/field in different directions (the field of the source is dependent in amplitude and orientation on the position of the individual sensor in relation to the source), noise in the background field can be further suppressed (fluctuations orthogonal to the measurement signal are suppressed). This allows for superior noise reduction compared to the conventional gradiometer approach.

For example, M1 to Mn are at least one magnetic field component and Z1 to Zn are at least one interference component (e.g. geomagnetic field), which can be static or variable over time. The corresponding NV magnetometer units S1 to Sn (expandable as shown in FIG. 1) measure the resulting components R1 to Rn, which contain the interference signal. In this process, NV magnetometer unit S1 measures the component R1, consisting of the first disturbance Z1 and the signal M1 at the location of S1, and NV magnetometer unit S2 measures the component R2, consisting of the second disturbance Z2 and the signal M2 at the location of S2, etc. From this, a signal that is insensitive to the disturbance components can be determined by vector arithmetic.

FIG. 3 shows a schematic block diagram of a measurement principle for NV vector gradiometry, as it can be used in an embodiment of the invention, for two NV magnetometer units S1 and S2. An evaluation can proceed as follows:

Two vector-valued signals R1 and R2 are measured. These contain signal components M1 and M1′ as well as interference components Z1 and Z2=Z1, which are the same for the measuring ranges in particular. The gradiometer measurement signal is then given by:

$$\vec{S} = \vec{R 1} - \vec{R 2} = ( \begin{matrix}{R}_{1 , x} \\ {R}_{1 , y} \\ {R}_{1 , z}\end{matrix} ) - ( \begin{matrix}{R}_{2 , x} \\ {R}_{2 , y} \\ {R}_{2 , z}\end{matrix} ) = ( \begin{matrix}{M}_{1 , x} + {Z}_{1 , x} \\ {M}_{1 , y} + {Z}_{1 , y} \\ {M}_{1 , z} + {Z}_{1 , z}\end{matrix} ) - ( \begin{matrix}{M}_{1 , x}^{′} + {Z}_{1 , x} \\ {M}_{1 , y}^{′} + {Z}_{1 , y} \\ {M}_{1 , z}^{′} + {Z}_{1 , z}\end{matrix} ) = ( \begin{matrix}{M}_{1 , x} + {M}_{1 , x}^{′} \\ {M}_{1 , y} + {M}_{1 , y}^{′} \\ {M}_{1 , z} + {M}_{1 , z}^{′}\end{matrix} ) . \qquad ( 1 )$$

As described by equation (1), the signal no longer contains any interference components, provided that these are uniform for the measuring ranges. Furthermore, the measurement signal is vectorial, which means that it is possible to determine the direction (“where does the signal come from?”), distinguish between different signals, and correlate several signals from different sensors in one arrangement (imaging method).

The result can be expressed in equation 1 using a magnitude and a direction. This is particularly advantageous if the source lies between the magnetometer units (or in a plane that passes through the center of the two) and thus the magnetic field is in the opposite direction.

By utilizing three or more magnetometer units (geometric arrangement), a system of equations can be set up that allows the individual magnetic fields at the magnetometer units to be determined.

Another advantage is that in conventional gradiometers, the difference between two scalar sensors is measured, which are connected in series. The performance of this depends on the fact that a sensor only measures the background field (measured values (scalar) are only subtracted from each other). Furthermore, in this case, the difference between the sensors is always smaller than the actual measured variable (<M). The approach described here can determine the measurement signal at different angles to the background signal by arranging the signals in different spatial angles (also when projected onto a plane). This increases the signal-to-noise ratio and thus the sensitivity (in the symmetrical ideal case, the magnetic field is exactly opposite with two sensors).

Another advantage of a vector gradiometer is that it can be built significantly smaller than a conventional gradiometer. Here, a reference sensor is arranged in such a way that its distance to the signal source is so great that the signal at the reference sensor is significantly smaller. This requires that the distance between the sensors is significantly greater than the distance between the source and the measuring sensor. By utilizing the vector information, all magnetometer units can be placed close to the source and no remote reference sensor is needed.

A “map” can be generated, i.e. a spatial resolution of the measurement signal (3D), by means of a calibration, an assumption regarding the distances to the signal origin or a reference sensor far away from the source, which characterizes the background field. Wherein a 2D resolution is intrinsically given by a sensor array. If no sensor array is used in a plane, a 2D resolution can be realized.

The distance d of the sensor heads within a gradiometer unit, i.e., in this case the distance of the two sensor diamonds, may be in the range of mm to a few cm, for example between 0.5 cm and 2 cm. For values in this order of magnitude, good results could be achieved, for example, for the measurement of biomagnetic fields, which are in the picotesla range. However, as long as sufficient independence of the signals from the background fields is achieved, larger or smaller distances or other gradiometer configurations can be used, such as sensors arranged next to each other. If the distance between the individual sensor heads is too large, the background field can no longer be identical at both locations, especially in the case of local background fields (electric lines, etc.). If the distance between the sensor heads is too small, however, the difference between the field strengths of the magnetic field to be measured between the two locations may be too small. The choice of the appropriate distance between the two sensors of a gradiometer unit is therefore also dependent on the type and orientation of the magnetic field source of interest and on the expected field strength. The distance between the sensor unit and the magnetic field source can also be in the mm to cm range.

In this case, signal processing has the task of making it independent from external magnetic field interference variables. Wherever electrical charges move or magnetic or magnetizable materials are moved, the ambient noise changes. This must be separated from the signal by appropriate signal processing in order to make the signal measurable.

In addition, particularly good signal processing enables improved signal resolution and thus the detection of rarer and more difficult to detect artifacts and associated clinical pictures.

Many environmental signals occur at a certain frequency. Signals can be seen in the power grid, particularly at 50 Hz or 60 Hz and higher harmonics. Filtering out these typical frequencies is highly advantageous, as it greatly reduces noise. In particular, the performance of gradiometer units can be significantly improved. For example, a notch filter or band-reject filter can be used at the frequency of an AC power supply (e.g. 50 Hz) and/or harmonics (e.g. 100 Hz, 150 Hz, etc.) to filter the measured signal.

Furthermore, high-pass filters can be used to advantage to filter out particularly low frequencies, e.g. below 1 Hz, and/or low-pass filters to filter out particularly high frequencies, e.g. above 200 Hz, from the measured signal. Particularly high and particularly low frequencies are those typically found in a cardiac signal (i.e., typically about 60 to 180 Hz)

In addition, further specific filters can be introduced which, when adapted to a particular environment, filter out interference frequencies, e.g. field frequencies or field patterns that typically occur in technical devices, for example. This can also be different for different applications. For example, interference frequencies in hospitals differ from those in private households.

1. A device for detecting magnetic signals generated by a beating heart, comprising:
at least two nitrogen-vacancy center (“NV”) magnetometer units arranged in a geometric configuration with respect to one another and connected to a signal processing unit,
wherein the device is configured:
to detect a magnetic field strength and field direction using each of the at least two NV magnetometer units, and
to determine using the signal processing unit at least one field vector comprising an effective magnetic field strength and an effective field direction from the magnetic field strengths and field directions detected by the at least two NV magnetometer units.

2. The device according to claim 1, wherein the geometric arrangement is a two-dimensional arrangement in which the at least two NV magnetometer units are arranged in a plane.

3. The device according to claim 1, wherein;
the at least two NV magnetometer units comprise at least four NV magnetometer units, and
the geometric arrangement is a three-dimensional arrangement in which at least one of the at least four NV magnetometer units is not arranged in a plane in which at least three others of the at least four NV magnetometer units are arranged.

4. The device according to claim 1, wherein:
each of the at least two NV magnetometer units has, as a sensor medium, a diamond crystal, or a section of a diamond crystal with nitrogen-vacancy centers, and
the device is configured to detect the magnetic field strength and the field direction by reading a magnetic resonance dependent on the magnetic field strength in the sensor medium.

5. The device according to claim 4, further comprising:
at least one excitation light source configured to radiate light into the sensor medium;
at least one microwave source configured to generate a resonant field in the sensor medium; and
at least one photodetector configured to detect resonance-dependent fluorescent light from the sensor medium.

6. The device according to claim 5, wherein a same excitation light source and/or a same microwave source are associated with at least two NV magnetometer units.

7. The device according to claim 4, wherein the sensor medium of the at least two NV magnetometer units each has a section of a same diamond crystal.

8. The device according to claim 4, wherein a distance between the sensor media of the at least two NV magnetometer units is from between 1 to 30 millimeters.

9. The device according to claim 4, further comprising:
at least one magnetic field device configured to generate a substantially homogeneous bias magnetic field at a location of the sensor media of the at least two NV magnetometer units,
wherein the bias magnetic fields of different NV magnetometer units are different.

10. The device according to claim 1, further comprising:
a band-elimination filter or notch filter having a filter frequency at a frequency of an AC power supply or a harmonic thereof, and/or a low-pass filter and/or a high-pass filter for filtering a detected signal.

11. The device according to claim 1, wherein the signal processing unit is configured to calculate a plurality of field vectors, each comprising a magnetic field strength and a field direction, from magnetic field data detected using the at least two NV magnetometer units.
